(19) United States
(12) Patent Application Publication
(10) Pub. No.: US 2024/0074648 A1
(43) Pub. Date: Mar. 7, 2024

(54) LEAK PREVENTION DEVICES, SYSTEMS, AND METHODS FOR ENDOSCOPE SYSTEMS

(71) Applicant: Boston Scientific Scimed, Inc., Maple Grove, MN (US)

(72) Inventors: Ryan V. Wales, Northborough, MA (US); Colby Harris, Norfolk, MA (US); Ryan Vincent William Pollock, Leominster, MA (US); Brittany Elizabeth Reed, Brighton, MA (US); Aakash Deora, Marlborough, MA (US)

(73) Assignee: Boston Scientific Scimed, Inc., Maple Grove, MN (US)

(21) Appl. No.: 18/459,285

(22) Filed: Aug. 31, 2023

Related U.S. Application Data
(60) Provisional application No. 63/374,334, filed on Sep. 1, 2022.

(51) Int. Cl. A61B 1/12; A61B 1/00; F16K 7/06

(52) U.S. Cl. CPC A61B 1/125; A61B 1/00128; F16K 7/06

(57) ABSTRACT

Devices, systems, and methods for mitigating leakage from connectors and tubing of an endoscope system when the connectors and or tubing are disconnected from fluid ports of the endoscope system. A tubing assembly for connecting a fluid line to an endoscope and a fluid source may include an elongate tube having a lumen, a connector coupled to an end of the elongate tube and having a port in fluid communication with the lumen, and a fluid control component configured to selective adjust fluid flow through the lumen and the port. The fluid control component may be located in one or more of a cover for the fluid source, the tubing, the connector, and/or other components in fluid communication with the lumen. The fluid control component may be a valve. The valve may be manually actuated from a closed position to an opened position.

### CROSS-REFERENCE TO RELATED APPLICATIONS

This application claims the benefit of U.S. Provisional Patent Application Ser. No. 63/374,334 filed on Sep. 1, 2022, the disclosure of which is incorporated herein by reference.

### TECHNICAL FIELD

This disclosure relates generally to leak prevention assemblies and methods, and particularly to leak prevention devices, systems, and methods for an endoscope system.

### BACKGROUND

A wide variety of intracorporeal and extracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In a first example, a tubing assembly for connecting to a fluid line of an endoscope and a fluid source may comprise an elongate tube, the elongate tube having a lumen, a connector coupled to an end of the elongate tube, the connector having a port in fluid communication with the lumen, and a fluid control component configured to selectively adjust fluid flow through the lumen and the port.

Alternatively or additionally to any of the examples above, in another example, the fluid control component may be configured to selectively depressurize the lumen.

Alternatively or additionally to any of the examples above, in another example, the fluid control component may be a valve configured to selectively adjust fluid flow through the lumen.

Alternatively or additionally to any of the examples above, in another example, the tubing assembly may further include a cover configured to couple to the fluid source, and wherein the valve may be in fluid communication with the fluid source through the cover and the valve is configured to be selectively actuated from a location exterior of the fluid source.

Alternatively or additionally to any of the examples above, in another example, the valve may be in fluid communication with the lumen through the connector and the valve is configured to be selectively actuated from a location exterior of the connector.

Alternatively or additionally to any of the examples above, in another example, the valve may be in the port and is configured to be actuated in response to the port coupling with an umbilicus of an endoscope.

Alternatively or additionally to any of the examples above, in another example, the valve may be biased to a closed position and configured to adjust from the closed position to an opened position in response to coupling the connector with fitting of the endoscope.

Alternatively or additionally to any of the examples above, in another example, the fluid control component may include a clamp configured to selectively engage the elongate tube.

Alternatively or additionally to any of the examples above, in another example, the elongate tube may include a bite valve in communication with the lumen and the bite valve blocks fluid flow through the lumen when in a resting configuration, and the clamp may be configured to engage the bite valve through the elongate tube to adjust the bite valve to an open configuration that allows fluid to flow through the lumen.

Alternatively or additionally to any of the examples above, in another example, the clamp may be configured to block fluid flow through the lumen when engaging the elongate tube.

Alternatively or additionally to any of the examples above, in another example, the lumen may be a first lumen and the elongate tube comprises a second lumen, the first lumen and the second lumen may be coaxial, and the fluid control component may be configured to selectively adjust fluid flow through the first lumen, the second lumen, and the port.

In a further example, a tubing assembly for connecting to a fluid line of an endoscope and a fluid source may comprise an elongate tube, the elongate tube having a lumen, a connector coupled to an end of the elongate tube, the connector in fluid communication with the lumen, a port in fluid communication with the lumen, the port is configured to couple to the fluid line of the endoscope, and a valve at the connector and in fluid communication with the lumen, the valve is configured to adjust between a closed position at which fluid through the valve is blocked and an opened position at which fluid flows through the valve.

Alternatively or additionally to any of the examples above, in another example, the valve may be configured to vent fluid to atmosphere when in the opened position and the connector is coupled to the endoscope or the fluid source.

Alternatively or additionally to any of the examples above, in another example, the valve may be configured to fluidly connect the fluid line of the endoscope to the lumen when the valve is adjusted from the closed position to the opened position.

Alternatively or additionally to any of the examples above, in another example, the valve may be biased to the closed position.

In a further example, a tubing assembly for connecting to a fluid line of an endoscope and a fluid source may comprise an elongate tube, the elongate tube having a lumen, a connector coupled to an end of the elongate tube, the connector in fluid communication with the lumen, a port in fluid communication with the lumen, the port is configured to couple to the fluid line of the endoscope, and a clamp configured to engage the elongate tube.

Alternatively or additionally to any of the examples above, in another example, the elongate tube may include a bite valve in communication with the lumen and the bite valve blocks fluid through the lumen when in a resting configuration, and the clamp is configured to engage the bite valve through the elongate tube to adjust the bite valve to an opened configuration that allows fluid to flow through the lumen.

Alternatively or additionally to any of the examples above, in another example, the clamp may be configured to block fluid flow through the lumen when engaging the elongate tube.

Alternatively or additionally to any of the examples above, in another example, the clamp may include a ratchet component configured to releasably lock the clamp in a position relative to the elongate tube.

Alternatively or additionally to any of the examples above, in another example, the clamp may extend from the connector and is configured to engage the elongate tube at a location proximal of the connector.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts a schematic view of an illustrative endoscope system, wherein the endoscope system is activated to deliver air to atmosphere;

FIG. 3B depicts a schematic view of an illustrative endoscope system, wherein the endoscope system is activated to deliver air to a patient through the patient end of the endoscope;

FIG. 3C depicts a schematic view of an illustrative endoscope system, wherein the endoscope system is activated to deliver lens wash fluid through the patient end of the endoscope;

FIG. 3D depicts a schematic view of an illustrative endoscope system, wherein the endoscope system is activated to deliver irrigation fluid through the patient end of the endoscope;

FIG. 4 depict a schematic view of an illustrative endoscope system;

FIG. 5 depicts a schematic side view of an illustrative connector;

FIG. 6 depicts a schematic cross-section view of an illustrative connector coupled to tubing;

FIG. 7 depicts a schematic cross-section view of an illustrative connector coupled to tubing;

FIG. 8 depicts a schematic cross-section view of an illustrative fluid reservoir;

FIG. 9A depicts a schematic side view of a connector with an illustrative fluid control component and coupled to tubing;

FIG. 9B depicts a schematic end view of the connector depicted in FIG. 9A taken at a cross-section of the tubing along line 9B-9B, wherein the fluid control component is in a relaxed position;

FIG. 9C depicts a schematic end view of the connector depicted in FIG. 9B, wherein the fluid control component is in an actuated position;

FIG. 10 depicts a schematic end view of an illustrative fluid control component engaged on tubing, wherein the tube is shown in cross-section;

FIG. 11A depicts a schematic top view of a connector with an illustrative fluid control component and coupled to tubing;

FIG. 11B depicts a schematic end view of the connector depicted in FIG. 11A taken at a cross-section of the tubing along line 11B-11B, wherein the fluid control component is in a relaxed position;

FIG. 11C depicts a schematic end view of the connector depicted in FIG. 11B, wherein the fluid control component is in an actuated position;

FIG. 12A depicts a schematic cross-section view of a connector having a fluid control component, wherein the connector is spaced from fluid ports;

FIG. 12B depicts a schematic cross-section view of the connector of FIG. 12A coupled with the fluid ports;

FIG. 13A depicts a schematic cross-section view of an illustrative port of a connector spaced from a fluid port; and

FIG. 13B depicts a schematic cross-section view of the illustrative port of FIG. 13A coupled with the fluid ports.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

An endoscope is used in performing diagnostic and/or therapeutic treatments by inserting an elongated shaft of the endoscope into a subject to observe a part to be examined within a body cavity of the subject and, if necessary, inserting a treatment instrument/tool into a working channel in the elongated shaft of the endoscope. Such endoscopes or endoscope systems may include a fluid/lens wash capability, or the like, configured to feed fluid such as gas (e.g., air, CO₂) to an end of the endoscope for insufflating the inside of the subject at a target site. Lens wash features may provide sterilized water at relatively high pressure to spray across and clear debris from a camera lens of the endoscope. In order to rinse the target site of the subject, separate from the air/water feed capability, endoscopes or endoscope systems may have an irrigation capability that provides lower pressure, higher volume water, supplied via a pump (e.g., peristaltic pump) to the target site in order to clear the field of view for observation and treatment. A water source (e.g., a fluid source) for lens wash and/or irrigation features may include one or more fluid reservoirs having tubing and cap assemblies that create a plumbing circuit in connection with the endoscope channels, valving, and/or connectors to accomplish the gas and water functions described.

Such tubing and cap assemblies may be available in various configurations, which may include a water bottle, a cap fitted for the specific bottle, and an array of tubing that is extendable through openings in the cap. The tubing typically is arranged to accommodate a specific configuration of endoscope fittings and valving, which does not tend to be modular or optional. In some cases, one or more connectors may be utilized to connect tubing for irrigation, lens wash, and/or insufflation features to an endoscope umbilical in fluid communication with working channels of the endoscope.

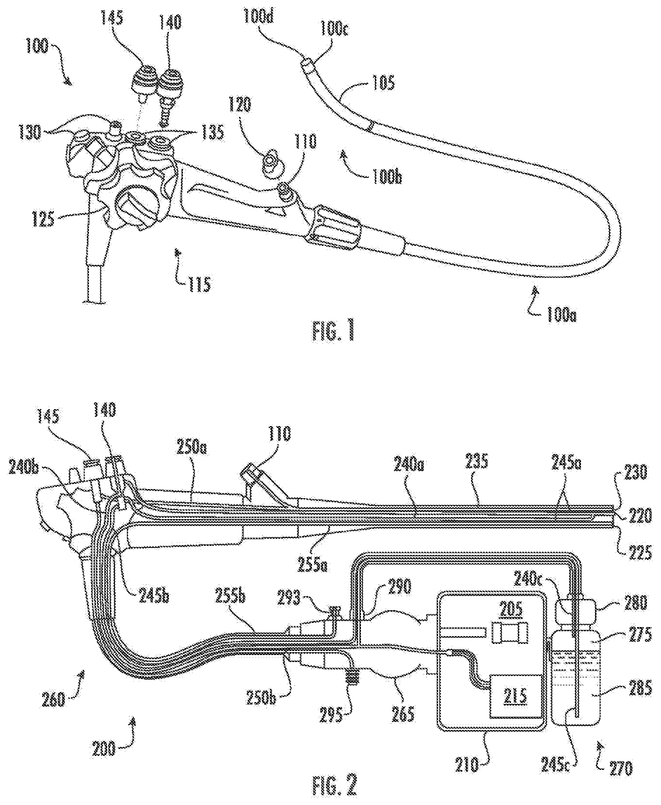

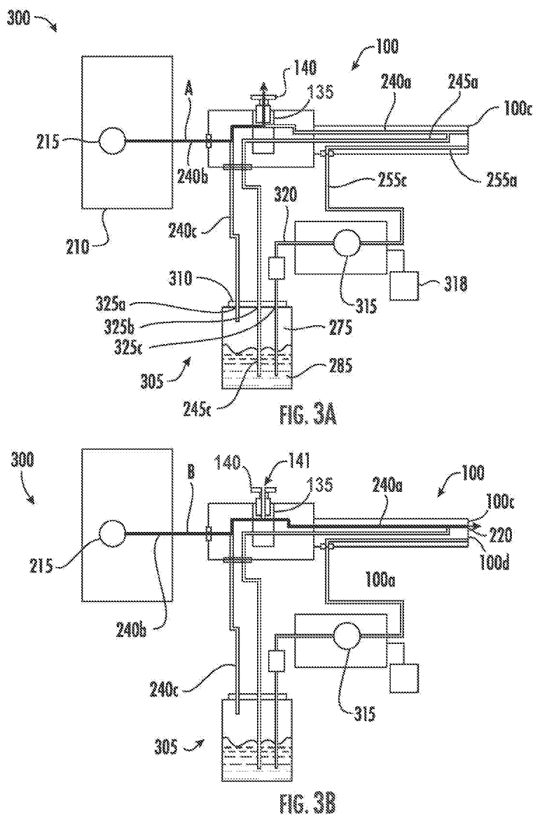

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient). Details of the endoscope 100 and the endoscope system 200 may be more fully described in U.S. Patent Application Publication No. 2022/0192479 A1, filed on Dec. 21, 2021, and titled TUBING ASSEMBLIES AND METHODS FOR FLUID DELIVERY, which is hereby incorporated by reference in its entirety for all purposes.

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., a lamp) may be located in a video processing unit 210 that processes signals input from the imager and outputs processed video signals to a video monitor (not shown) for viewing. The video processing unit 210 may also serve as a component of an air/water feed circuit by housing a pressurizing or air pump 215, such as an air feed pump, in the unit 210. Other suitable pumps for the air/water feed circuit are contemplated.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the subject at the treatment area and for supplying water to wash a lens covering the imager. An irrigation opening 225 in the end face 100d supplies irrigation fluid to the treatment area of the subject. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the flexible bending portion 105 (e.g., one knob may control up-down steering and another knob may control left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 may have a gas (e.g., air or CO₂) feed line 240b, a lens wash feed line 245b, a suction feed line 250b, an irrigation feed line 255b, a light guide (not shown), an electrical signal cable (not shown), and/or other suitable lines, guides, and/or cables. The connector portion 265 when plugged into the video processing unit 210 connects the light source 205 in the video processing unit with the light guide. The light guide may run along the umbilical 260 and the length of the endoscope shaft 100a to transmit light to the distal tip 100c of the endoscope 100. The connector portion 265, when plugged into the video processing unit 210, may also connect the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c may pass from one end positioned in an air gap 275 between the top 280 (e.g., a bottle cap) of the reservoir 270 and the remaining water 285 (e.g., the remaining water 285) in the reservoir to a connector 290 on the outside of the connector portion 265. The gas feed line 240b from the umbilical 260 branches in the connector portion 265 to fluidly communicate with the gas supply tubing 240c at the detachable connector 290, as well as the air pump 215. A length of lens wash tubing 245c, with one end positioned at the bottom of the reservoir 270, may pass through the top 280 of the reservoir 270 to the same detachable connector 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some configurations, irrigation water may be supplied via a pump (e.g., peristaltic pump) from a water source (not shown) independent from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve 140 in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

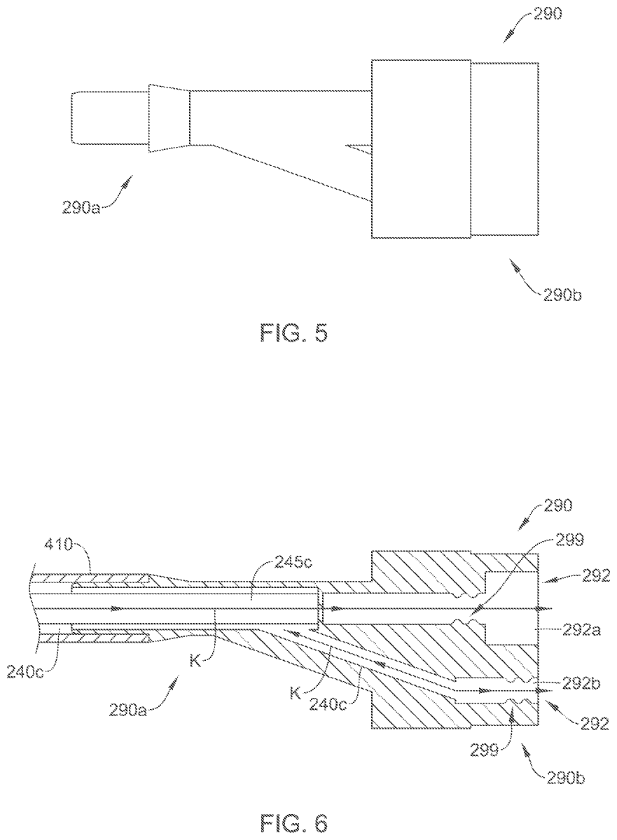

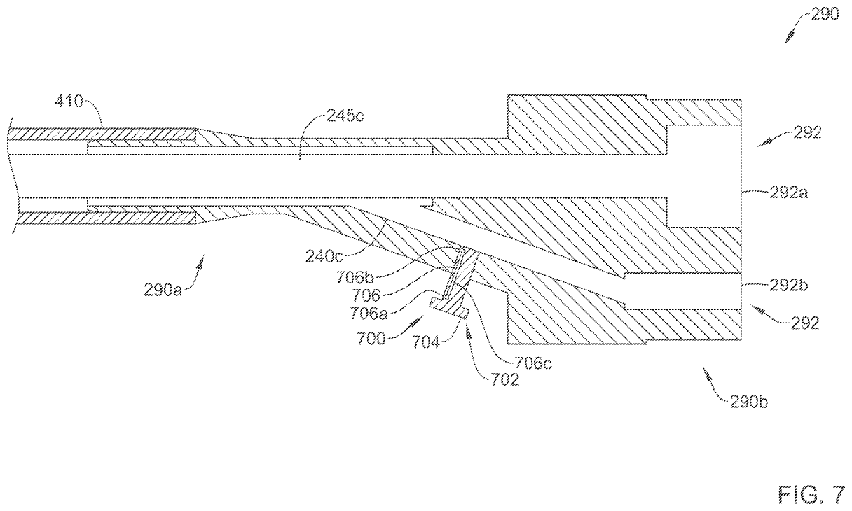

The gas supply tubing 240c and the lens wash tubing 245c may be combined in a coaxial relationship, but this is not required. In one example, the gas supply tubing 240c may define a lumen that is sufficiently large in diameter to encompass a smaller diameter lens wash tubing 245c, coaxially received within the gas supply tubing, as well as provide air to the water source in an annular space surrounding the lens wash tubing to pressurize the water reservoir (e.g., gas tubing 240c and lens wash tubing 245c as configured in the connector 290 depicted in FIG. 6). The lens wash tubing 245c may be configured to exit the lumen defined by the coaxial gas supply tubing in any suitable sealed manner, such as for example an aperture, fitting, collar, and/or the link for the purpose of transitioning from the coaxial arrangement to a side-by-side arrangement of the detachable gas/lens wash connection to the endoscope connector portion 265. In one example, a fitting for such a transition is depicted as the connector 290 in FIGS. 5 and 6 (e.g., a coaxial split connector, but other suitable connector configurations are contemplated).

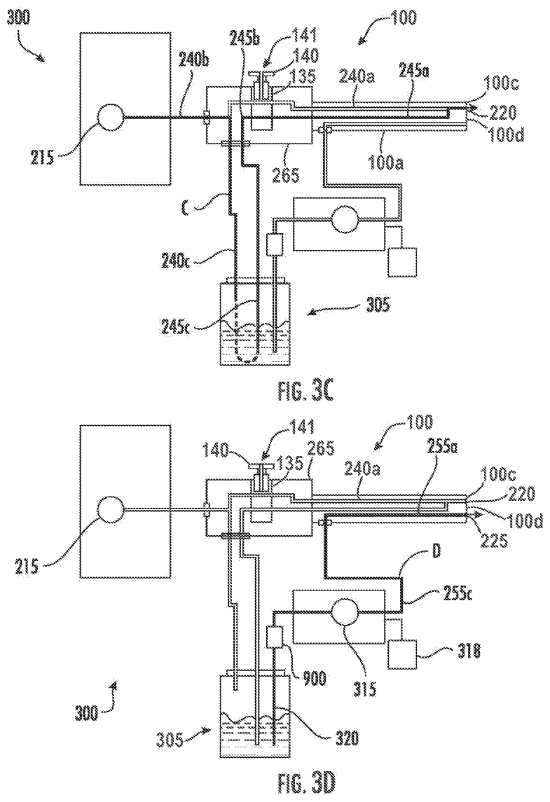

FIGS. 3A-3D are schematic views illustrating an operation of an embodiment of an endoscope system 300, which may be similar or dissimilar to the endoscope system 200, where the supply tubing for irrigation and lens wash are connected to and drawn from a single water reservoir 270, 305. The hybrid system 300 may include the single water reservoir 270, 305, a cover or cap 310 for the reservoir, gas supply tubing 240c, lens wash tubing 245c, irrigation pump 315 that may be in communication with foot switch 318 or other suitable switch, upstream irrigation tubing 255c, 320, and downstream irrigation supply tubing 255c.

The cap 310 may be configured to attach in a seal-tight manner to the water reservoir 270, 305 by a threaded arrangement and/or other suitable coupling mechanism. The cap 310 may include a gasket to seal the cap 310 to the reservoir 270, 305. The gasket may be an O-ring, flange, collar, and/or the like and can be formed of any suitable material. A number of through-openings (325a, 325b, 325c) in the cap 310 may be provided to receive, respectively, the gas supply tubing 240c, lens wash tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir. The lens wash tubing 245c may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265 (e.g., FIG. 2). An example of a suitable connector for such transition is the connector 290 depicted in FIGS. 5 and 6.

In various embodiments, different configurations of valving may be incorporated into the tubing of the system 200, 300. For example, an in-flow check valve may be disposed in the path of the gas supply tubing 240c to help prevent backflow into the air pump 215. In this manner, pressure building within the water reservoir 270, 305 may create a pressure difference between the water reservoir 270, 305 and the gas supply tubing 240c helping to maintain a positive pressure in the water reservoir 270, 305 even when large amounts of water may be removed from the water source during the irrigation function. This arrangement may compensate for any time lag in air being delivered from the air pump 215 to the water reservoir 270, 305, which might otherwise cause a negative pressure vacuum in the water reservoir 270, 305. Similarly, an out-flow check valve, such as a one-way valve, may be incorporated in the lens wash tubing 245c, upstream irrigation supply tubing 320, and/or downstream irrigation supply tubing 255c to help prevent backflow of water from either or both of the lens wash and irrigation tubing in the event of a negative pressure situation, as described.

More generally, in some configurations, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check, a flapper valve, and/or other suitable check valve. Accordingly, a check valve as used herein may be meant to be separate and distinct from an active valve that is operated in a binary manner as an on/off valve or switch to allow flow to be turned on or allow flow to be turned off (e.g., a stop cock valve, solenoid valve, peristaltic pump, blow off valve).

During operation of the system of FIGS. 3A-3D, a flow of water for irrigation may be achieved by operating the irrigation pump 315 via the foot switch 318 and/or other suitable actuation mechanism. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 270, 305, the pressure in the system may be controlled to maintain the lens wash tubing 245c at substantially the pressure necessary to accomplish a lower flow rate lens wash, while compensating for reduced pressure in the water reservoir 270, 305 due to supplying a high flow rate irrigation. When pressure is reduced in the water reservoir 270, 305 by use of the lens wash function, the irrigation function, or both functions simultaneously, the reduced pressure may be compensated for by the air pump 215 via the gas supply tubing 240c.

Flow paths in the schematic set-ups depicted in each of FIGS. 3A-3D have been highlighted to show the different flow paths possible with the hybrid system 300 having supply tubing (e.g., irrigation tubing 255c, 320, lens wash tubing 245c and/or other suitable tubing) connected to and drawn from the single water reservoir 270, 305. Not all features depicted in each of FIGS. 3A-3D are labeled with reference numerals in each of FIGS. 3A-3D for clarity purposes, but similarly depicted features in FIGS. 3A-3D should be understood to be referring to a same or similar feature in each of FIGS. 3A-3D.

As shown in FIG. 3A, the endoscope 100 may be in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b (e.g., in the umbilical 260 via the connector portion 265, as depicted in FIG. 2) and through the gas/water valve 140 to atmosphere. Because the system is open at the vent hole in the gas/water valve 140, there is no build up to pressurize the water reservoir 270, 305 and consequently no water is pushed through the lens wash tubing 245c.

As shown in FIG. 3B, the endoscope 100 may be in a gas delivery state with the gas/water valve 140 in a first position. When gas is called for at the distal tip 100c, for example, to clean the end face 100d of the distal tip 100c or insufflate the patient body in the treatment area, the user may close off a vent hole 141 in the gas/water valve 140 with a thumb, finger, or the like (first position). In this state, gas (pressure) may be delivered along path B from the air pump 215 and flowed through the gas feed line 240b (e.g., in the umbilical 260 via the connector portion 265, as depicted in FIG. 2). The gas may continue through the gas/water valve 140 to the gas supply line 240a in the endoscope shaft 100a and out the gas/lens wash nozzle 220 at the distal tip 100c. There is no build up to pressurize the water reservoir since the system is open at the gas/lens wash nozzle 220, and consequently no liquid is pushed through the lens wash tubing 245c.

As shown in FIG. 3C, the endoscope 100 may be in a lens wash delivery state with the gas/water valve 140 in a second position. When lens wash is called for at the distal tip 100c, for example, to clean the end face 100d of the distal tip 100c, the user, keeping the vent hole 141 in the gas/water valve 140 closed off, depresses the valve 140 to its furthest point in the valve well 135. The second position blocks off the gas supply to both atmosphere and the gas supply line 240a in the endoscope 100, and opens up the gas/water valve 140 to allow lens wash water to pass through to the lens wash line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 270, 305. The gas (pressure) pressurizes the surface of the remaining water 285 in the reservoir 270, 305 and pushes water up the lens wash tube 245c (e.g., to the connector portion 265 of the umbilical 26, as depicted in FIG. 2. The pressurized lens wash water may be pushed further through the lens wash feed line 245b and through the gas/water valve 140. Because the system 300 is closed, gas pressure may be allowed to build and maintain a calibrated pressure level in the water reservoir 270, 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user may activate the irrigation pump 315 (e.g., by depressing foot switch 318 or other suitable actuation mechanism) to deliver water or other liquid from the reservoir 270, 305 along path D. With the pump 315 activated, water is sucked out of the water reservoir 270, 305 through the upstream irrigation supply tubing 255c, 320 and pumped along the downstream irrigation supply tubing 255c to the connector portion 265. The irrigation pump head pressure pushes the irrigation water further through the irrigation feed line 255b (e.g., extending through the umbilical 260), through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

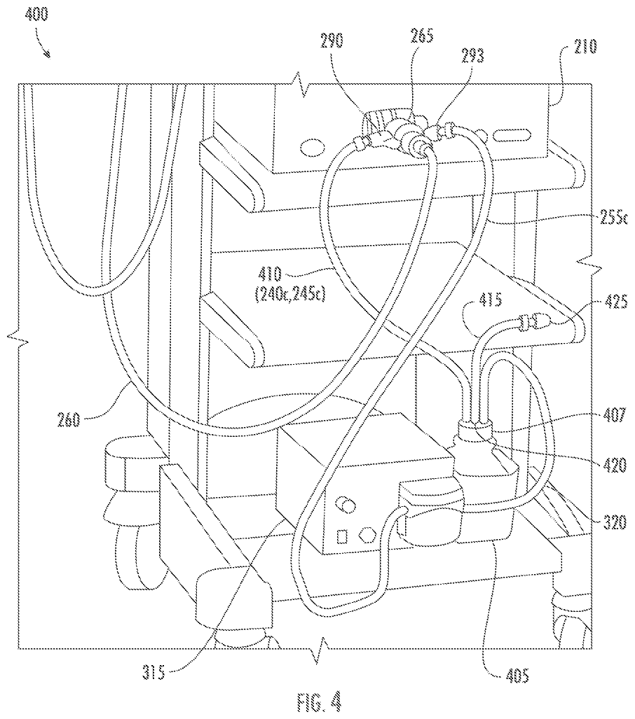

FIG. 4 is a schematic view illustrating a further configuration of a hybrid endoscope system 400 including a video processing unit 210, a connector portion 265, a peristaltic irrigation pump 315, a water reservoir 405 and a top or 407 on the reservoir 405, a coaxial gas and lens wash tubing 410, upstream and downstream irrigation supply tubing 255c, and alternative gas supply tubing 415 (e.g., CO2). A portion of the alternative gas supply tubing 415 passes from one end positioned in a gas gap (e.g., the gas gap 275, as depicted in FIG. 3A) between the top 407 (e.g., a cap or other suitable top) of the water reservoir 405 and the remaining water (e.g., the remaining water 285, as depicted in FIG. 3A) in the reservoir 405 through an additional opening 420 in the top 407 of the reservoir 405 to a detachable connection 425 for a source of the alternative gas supply (e.g., CO2 hospital house gas source). When the alternative gas supply is desired, such as CO2 gas, an air pump (e.g., the air pump 215 or other suitable air pump) on the video processing unit 210 and/or another suitable location may be turned off and CO2 gas, rather than air, is thereby flowed to the water reservoir 405 pressurizing the water surface. In the neutral state, CO2 gas flows backward up the gas supply tubing 240c to the connector portion 265, up the gas feed line (e.g., the gas feed line 240b or other suitable gas feed line), and is vented through a gas/water valve to atmosphere.

In the first position, the user may close off the vent hole in the gas/water valve, and the CO2 gas may be flowed through the gas/water valve to the gas supply line in the endoscope shaft and out the gas/lens wash nozzle at the distal tip 100c. In the second position, the user depresses the valve 140 to the bottom of the valve well, keeping the vent hole in the gas/water valve closed off. The second position blocks the CO2 gas supply to both atmosphere and the gas supply line 240a in the endoscope 100, and opens up the gas/water valve 140 to allow lens wash water to pass through to the lens wash supply line in the endoscope shaft and out the gas/lens wash nozzle at the distal tip. Gas (pressure) in the reservoir 405 may be maintained by delivery of gas through alternative gas (e.g., CO2) supply tubing 415. The irrigation function may be accomplished in a similar manner as the operation described above with respect to FIG. 3D and/or in one or more other suitable manners.

Referring to FIGS. 5 and 6, in one configuration, the connector 290 may be in the form of a coaxial split connector configured to couple to the coaxial tubing 410, either directly or indirectly, at a first end 290a (e.g., a proximal end) and the umbilical 260 (e.g., a connector 265 of the umbilical 260) of the endoscope system 200, 300, 400 at a second end 290b (e.g., a distal end). For example, in connection with the illustrated configuration in FIG. 6, the connector 290 may be coupled, directly or indirectly, to the endoscope end (e.g., a distal end) of coaxial tubing 410, so that the coaxial portions of the tubing 410 may be coupled to the endoscope 100 via the connector 290. In use, the connector 290 may allow for flow to transition between a coaxial arrangement and a side-by-side arrangement with flow paths K through ports 292 (e.g., a first port 292a for water and a second port 292b for gas), shown in FIG. 6. In some cases, the first port 292a and the second port 292b may be parallel to one another, but this is not required and other suitable relative positions of the first port 292a and the second port 292b are contemplated. Although the connector 290 is depicted as a coaxial split connector in FIG. 6, other suitable connectors 290 may be configured to connect parallel tubing or a single tube to the umbilical 260 of the endoscope system 200, 300, 400, as desired.

In some cases, the ports 292 may include one or more features configured to facilitate the connector 290 engaging a fluid port of the endoscope system 200, 300, 400. As depicted in FIG. 6, the ports 292 may include features such as coupling portions 299 (e.g., ribs, ridges, etc.) configured to engage a male feature of the fluid ports of the endoscope system. Alternatively or additionally, the one or more features configured to engage a fluid port of the endoscope system 200, 300, 400 may take on one or more other suitable configurations including, but not limited to, a one-way valve, a duck-bill valve, a flapper valve, and/or other suitable features configured to engage the fluid ports of the endoscope system 200, 300, 400.

In some cases and although other configurations are contemplated, the connector 290 may include a support structure component and an over molded or fluid device connecting component (e.g., which may include the ports 292). Example configurations of the connector 290 include, but are not limited to, the connector configurations described in Application No. 63/399,528, entitled TUBE CONNECTOR FOR ENDOSCOPE SYSTEMS filed on filed on Aug. 19, 2022, which is hereby incorporated by references in its entirety and for all purposes and U.S. Patent Application Publication No. 2022/0192479 A1, which has been incorporated, herein, by reference in its entirety and for all purposes.

When tubing, caps, and/or connectors used for irrigation, lens wash, and/or insufflation features of an endoscope may be disconnected from other components of the endoscope system after use (or prior to and/or during use) while a fluid reservoir coupled to the tubing, caps, and/or connectors is under pressure, water from the water reservoir may discharge from the tubing and/or the connectors in an undesirable manner due to the water being pressurized during use of the endoscope system. In some cases, the undesirably discharged or discharging of water and/or other fluids may result in a hazardous work environment for nurses, technicians, physicians, and/or other personnel in an operating or therapy room in which the endoscope system is used.

Various illustrative cap configurations, connector configurations, tubing configurations, and cap, connector, and/or tubing assemblies are discussed herein that are configured to address the problem of pressured and/or irrigation lines leaking water or other fluid when removed from connection with an endoscope umbilical by providing the assemblies with features to prevent or mitigate an amount of unintended fluid discharging from the tubing and/or connector when disconnected from the endoscope umbilical. In some examples, the reservoirs, caps, connectors, tubing, and/or assemblies thereof may be configured with valves, clasps, and/or other suitable features designed to prevent or mitigate unintended release of liquid and/or other fluid from the connectors and/or tubing. In some cases, the concepts discussed herein may facilitate flexibility in when and/or how connectors between tubing and an endoscope umbilical are coupled to the umbilical during procedures and may reduce steps for connecting and discussing connectors and/or tubing when using existing endoscope system configurations.

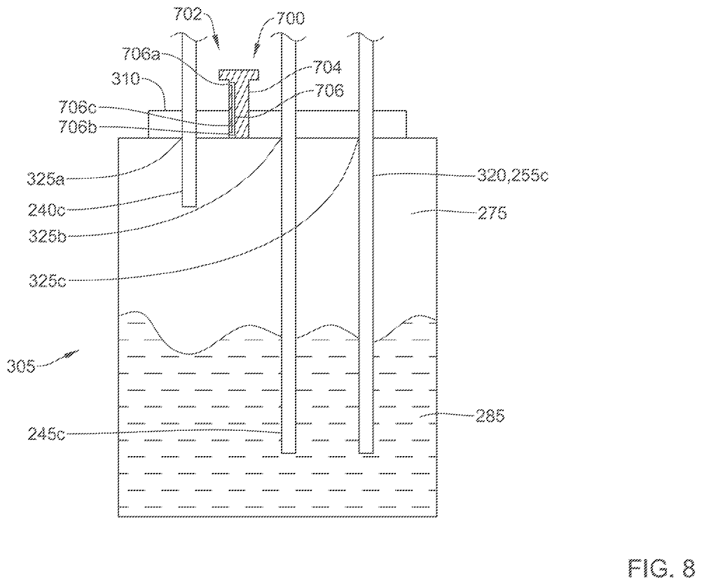

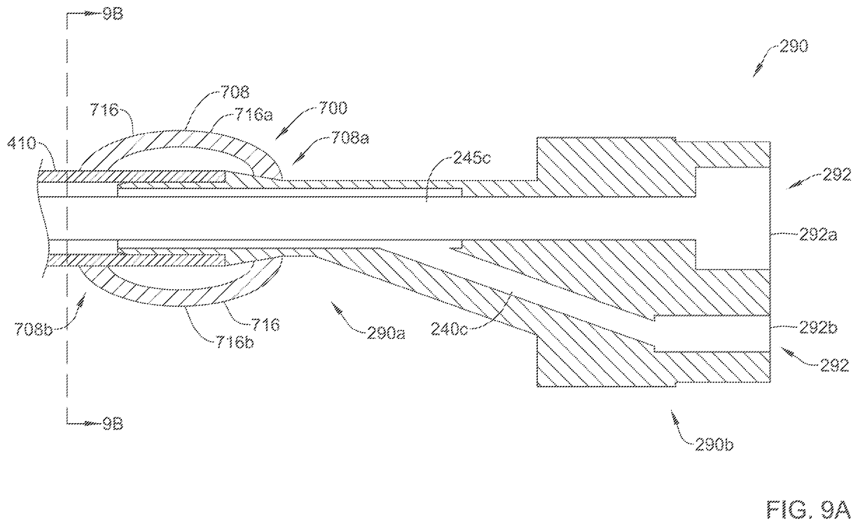

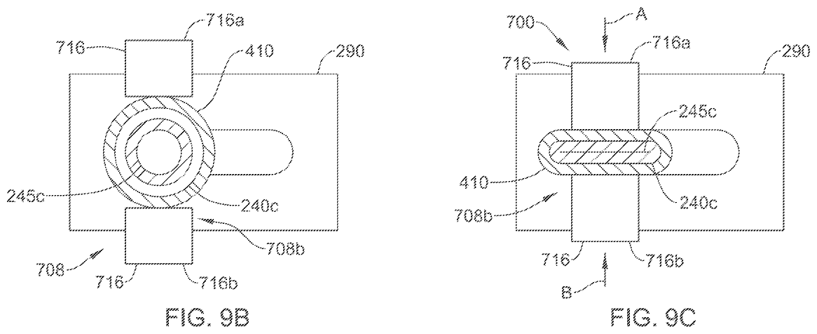

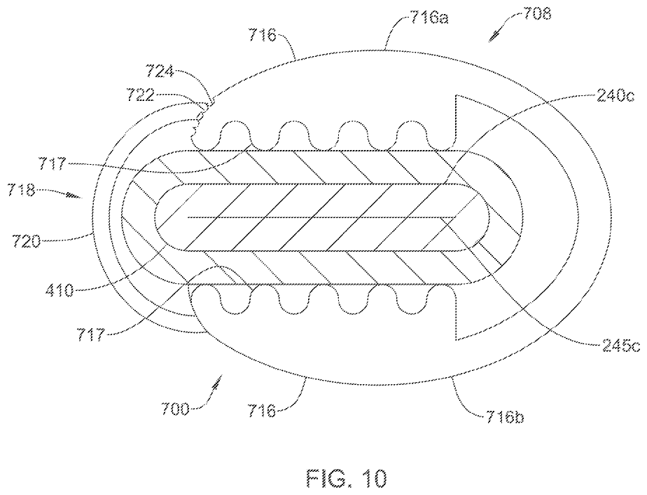

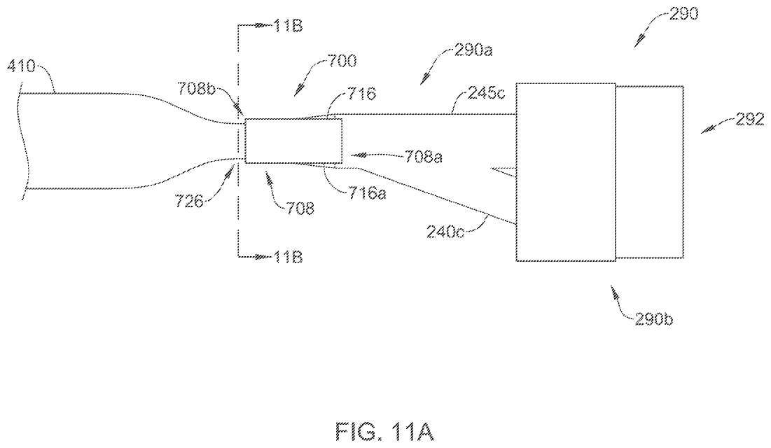

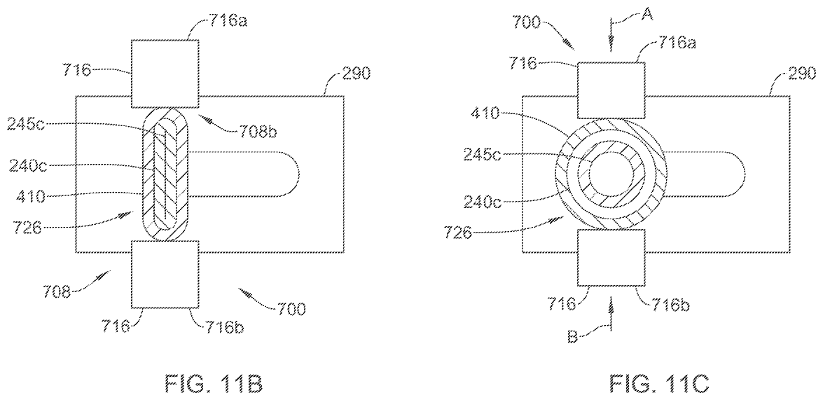

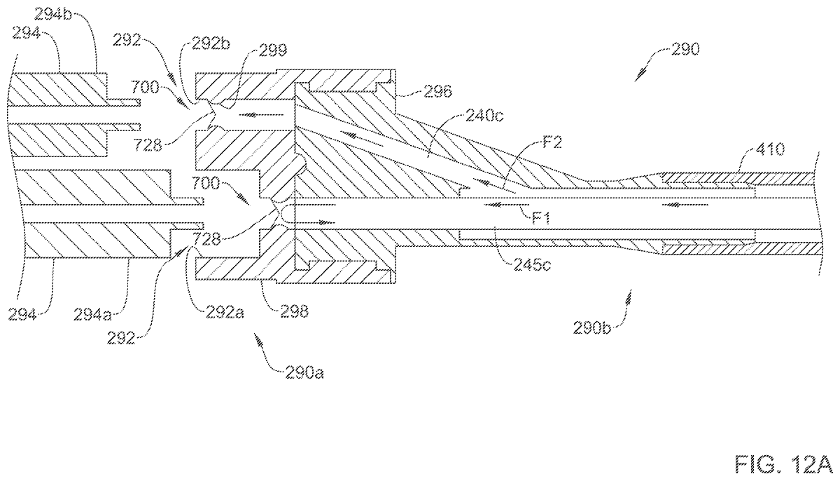

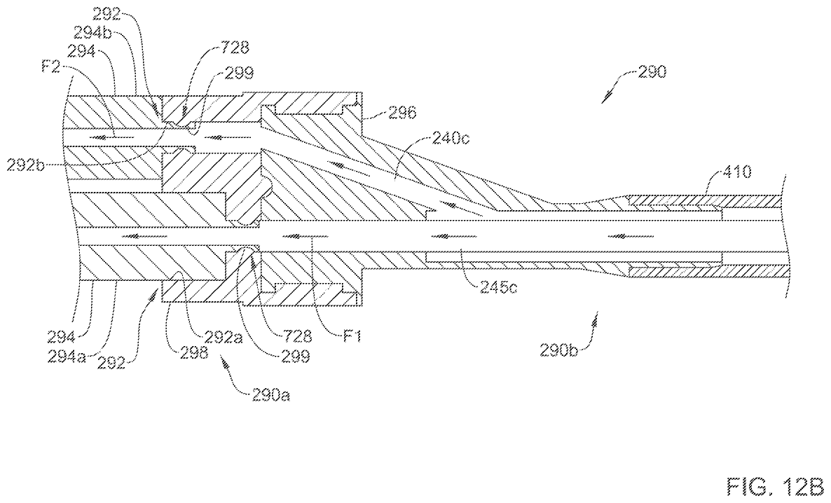

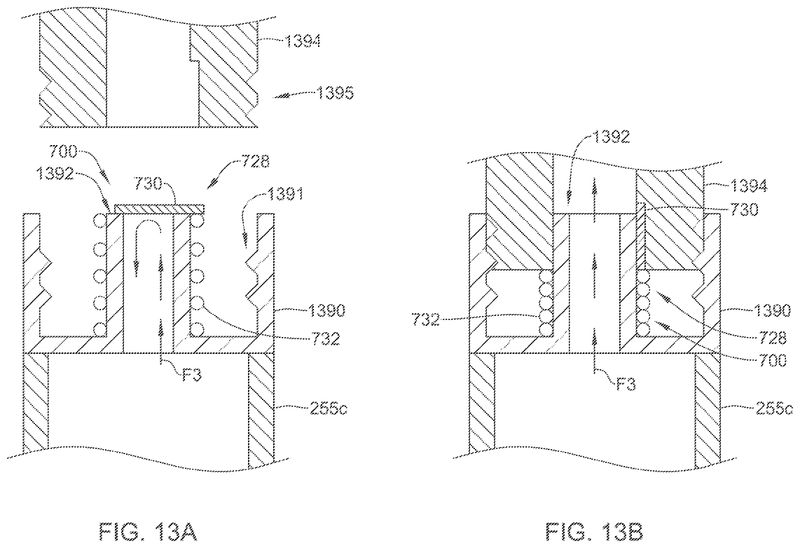

One or more components of the endoscope system 200 may include a flow control component 700. The flow control component 700 may be configured to selectively adjust flow through at least one lumen and/or port of the connector 290 and/or the irrigation supply tubing 255c, 320 to facilitate preventing leakage of liquid from components (e.g., the connector 290, the irrigation supply tubing 255c, etc.) of the endoscope system 200. Example flow control components 700 may include, but are not limited to, a blow-off valve (e.g., as depicted in FIGS. 7 and 8), a clip 708 (e.g., as depicted in FIGS. 9A-10), a bite valve 710 (e.g., as depicted in FIGS. 11A-11C), a one-way valve 712 (e.g., as depicted in FIGS. 12A and 12B), a spring valve (e.g., as depicted in FIGS. 13A and 13B), and/or other suitable flow control components.

As depicted in FIG. 7, the connector 290 may include the flow control component 700 configured to be selectively actuated from a location exterior of the connector 290 or from another location to depressurize a pressurized lumen and adjust fluid flow through the lumen. In some cases, the flow control component 700 may be in communication with the gas supply tubing 240c, which may be and/or may be in communication with a pressurized gas line that pressurizes a water reservoir in communication with the lens wash tubing 245c. As such, when in a resting or relaxed position, the flow control component 700 may allow the gas to flow through the gas supply tubing 240c unimpeded and allow the water reservoir to remain pressurized. When in an actuated position, the flow control component 700 may release gas from the gas supply tubing 240c, which in turn depressurizes the water reservoir in fluid communication with the gas supply tubing 240c. When the water reservoir is depressurized by actuating the flow control component 700 in communication with the gas supply tubing 240c, the water or other liquid in the lens wash tubing 245c in communication with the water reservoir will no longer be pressurized and unintended expelled or discharged water through the first port 292a of the connector 290 may be mitigated.

In some cases, the flow control component 700 may be a blow-off valve 702, but this is not required. The blow-off valve 702 may be any suitable type of valve configured to allow pressurized gas to be released (e.g., vented) to atmosphere or a less pressurized location. Example suitable types of blow-off valves 702 may include, but are not limited to, stock cock valves, pressure release valves, Shrader or American valves, and/or other suitable types of types of valves configured to release pressure upon actuation.

In some cases, the blow-off valve 702 may include an actuator 704 and a fluid passageway 706. The fluid passageway 706 may include a first opening 706a configured to be in communication with atmosphere, a second opening 706b configured to be in communication with fluid in the gas supply tubing 240c when in the actuator 704 is actuated, and a lumen 706c extending between the first opening 706a and the second opening 706b, as depicted in FIG. 7. Alternatively or additionally, the lumen 706c of the actuator 704 may be in selective communication with a lumen extending through the connector 290 to atmosphere. Other suitable configurations of the blow-off valve 702 are contemplated.

Further, the actuator 704 may be configured to have a first position and a second position. The first position of the actuator 704 may be a position at which at least one of the first opening 706a and the second opening 706b is not in communication with atmosphere or fluid in the gas supply tubing 240c, respectively, such that fluid does not flow through the lumen 706c. The second position of the actuator 704 may be a position at which the first opening 706a and the second opening 706b are in communication with atmosphere and fluid in the gas supply tubing 240c, respectively, such that fluid does flow from the gas supply tubing 240c into the second opening 706b, through the lumen 706c, and out of the first opening 706a. In some cases, the first position may be a closed or relaxed position of the actuator 704 and the second position may be an opened or actuated position of the actuator 704, but this is not required.

The actuator 704 may be configured to be actuated in any suitable manner. In one example, when the actuator 704 includes a threaded connection to a component (e.g., the connector 290, the cap 310, and/or other suitable component) of the endoscope system 200, 300, 400, the actuator 704 may be actuated by rotating the actuator 704 to cause the actuator 704 to adjust from the first position to the second position. In another example, when the actuator 704 is biased by a biasing mechanism (e.g., a spring, a bias of material (e.g., an over-mold material and/or other suitable material) of the connector 290, and/or other suitable biasing mechanism) to the first position, the actuator 704 may be actuated by applying a force to the actuator 704 and against a biasing force of the biasing mechanism to cause the actuator 704 to adjust from the first position to the second position. In the configuration depicted in FIG. 7, the actuator 704 may be actuated by rotation and/or by an application of force acting against a biasing force to cause the second opening 706b to be in fluid communication with the gas supply tubing 240c and thus, fluid to from the gas supply tubing 240c to enter the second opening 706b, pass through the lumen 706c, and out of the first opening 706a until pressure in the gas supply tubing 240c and a fluidly connected water reservoir has equalized with a pressure at atmosphere.

In an additional or alternative configuration, the blow off valve 702 in selective communication with the gas supply tubing 240c of the connector 290 may be formed in or by the material of the connector 290. In some cases, at least a portion of the connector 290 may be formed from a resilient material (e.g., thermoplastic elastomer (TPE), styrene-ethylene-butylene (SEB), rubber, silicone, and/or other suitable materials) and the blow-off valve may include a lumen extending through the resilient material, where the resilient material biases the lumen to the first position (e.g., closed or relaxed position) to block flow through the lumen between the gas supply tubing 240c and atmosphere and a user may squeeze or otherwise apply a force to the resilient material to adjust the lumen to the second position (e.g., an opened or actuated position) that allows fluid to flow through the lumen from the gas supply tubing 240c to atmosphere until pressure in the gas supply tubing 240c and the water reservoir equalizes with atmosphere.

The flow control component 700 depicted in FIG. 8 may be incorporated in the cap 310 on the water reservoir 270, 305, 405 (e.g., a fluid source) in communication with the gas supply tubing 240c, the lens wash tubing 245c, and/or the irrigation tubing 255c, 320. Although the flow control component 700 has been incorporated into the cap 310 in FIG. 8, the flow control component 700 may be incorporated into other components of or in communication with the water reservoir 270, 305, 405 and configured to remain above a water line in the water reservoir 270, 305, 405.

Although not required, the flow control component 700 depicted in FIG. 8 may take the form of the blow-off valve 702. When so configured, the blow-off valve 702 depicted in FIG. 8 may be configured to be selectively actuated (e.g., from exterior of the water reservoir 270, 305, 405 and/or otherwise actuated) to allow gas to flow from water reservoir 270, 305, 405 to atmosphere until a pressure in the water reservoir has equalized with pressure at atmosphere. When the flow control component 700 depicted in FIG. 8 takes the form of the blow-off valve 702, the blow-off valve 702 may take on similar configurations and/or operate in a manner similar to the configurations of blow-off valve 702 discussed herein (e.g., with respect to FIG. 7) to selectively reduce pressure in the water reservoir 270, 305, 405 by releasing gas to atmosphere.

Further, the blow off valve 702 in communication with the cap 310 or other suitable portion of the water reservoir 270, 305, 405 may be formed in or by the material of the cap 310 or other portions of the water reservoir 270, 305, 405. In some cases, at least a portion of the cap 310 may be formed from a resilient material (e.g., thermoplastic elastomer (TPE), styrene-ethylene-butylene (SEB), rubber, silicone, and/or other suitable materials) and the blow-off valve 702 may include a lumen extending through the resilient material, where the resilient material biases the lumen to the first position (e.g., closed or resting position) to block flow through the lumen between the gas supply tubing 240c and atmosphere and a user may squeeze or otherwise apply a force to the resilient material to adjust the lumen to the second position (e.g., an opened or actuated position) that allows fluid to flow through the lumen from the water reservoir 270, 305, 405 to atmosphere until pressure in the water reservoir 270, 305, 405 equalizes with atmosphere.

FIGS. 9A-C depict schematic views of an illustrative fluid control component 700 in the form of a clamp or clip 708 configured to engage the tubing 410, the lens wash tubing 245c, the gas supply tubing 240c, the irrigation tubing 255c, and/or other suitable tubing of the endoscope system 200, 300, 400. In some cases, the clamp or clip 708 may be adjusted (e.g., mechanically adjusted) from a first position to a second position to inhibit flow of fluid in or out of tubing of the endoscope system 200, 300, 400 and/or the connector 290.

The clamp or clip 708 may be selectively coupled to the tubing and/or one or more connectors of the endoscope system 200, 300, 400 or may be at least partially fixed with respect to the tubing and/or more connectors of the endoscope system 200, 300, 400. In one example, as depicted in FIG. 9A, the clamp or clip 708 may have a first end 708a coupled to the first end 290a of the connector 290 and may extend proximal from the connector 290 to a location over the coaxial tubing 410 at which a second end 708b of the clamp or clip 708 may engage the coaxial tubing 408. Although not depicted, the clamp or clip 708 may be similarly configured to engage other tubing of the endoscope system 200, 300, 400 including, but not limited to, irrigation supply tubing 255c.

The clamp or clip 708 may include arms 716. As depicted in FIGS. 9A-9C, the arms 716 of the clamp or clip 708 may include a first arm 716a and a second arm 716b. In operation, a user may apply force to the arms 716 to adjust the arms 716 from a first position at which fluid may flow through the lumens of the coaxial tubing 410 to a second position at which fluid may be blocked from passing through one or more of gas supply tubing 240c and the lens wash tubing 245c.

The arms 716 may terminate in surfaces that are configured to engage the coaxial tubing 410. Although not required, the surface configured to engage the coaxial tubing 410 may have any suitable size and/or shape that facilitates closing the lumens or tubes 240c, 245c. In one example, the surfaces configured to engage the coaxial tubing 410 may have a width sufficient to close the lumens of the coaxial tubing 410 and may have one or more sets of grips or teeth (e.g., grips or teeth 717 depicted in FIG. 10) configured to engage grip an outer surface of the tubing 410 while maintaining the tubing 410 in a second or closed position.

In some cases, the arms 716 may be biased toward one of the first position and the second position, but this is not required. When biased, the arms 716 may be biased using any suitable biasing technique described herein or otherwise, including, but not limited to, a spring, a spring constant of a resilient material of the tubing, a spring constant of a resilient material of the clamp or clip 708, and/or other suitable biasing mechanism.

The second end 708b of the clamp or clip 708 may be configured to engage the tubing (e.g., the tubing 410, 240c, 245c, 255c, and/or other suitable tubing) at any desired location. Although not required, the second end 708b of the clamp or clip 708 may be configured to engage the tubing at a location where the tubing is necked down or the layers of the tubing are thinner than at other locations along the tubing to facilitate adjusting the tubing from an opened position to a closed position.

FIG. 9B depicts a schematic end view of the clamp or clip 708 on the connector 290 and a cross-section view of the tubing 410 of FIG. 9A, taken along line 9B-9B. As depicted in FIG. 9B, the clip 708 and arms 716a and 716b are positioned in a first position such that fluid may flow through the gas supply tubing 240c and the lens wash tubing 245c.

FIG. 9C depicts a schematic end view of the clamp or clip 708 on the connector 290 and a cross-section of the tubing 410, with the clamp or clip in the second position such that flow is blocked from flowing through the lumens or tubing (e.g., the gas supply tubing 240c and the lens wash tubing 245c and/or other suitable tubing). In one example, a force in the direction of arrow A may be applied to the first arm 716a and/or in the direction of arrow B may be applied to the second arm 716b to adjust the clamp or clip 708 to the second position and block flow through the lumens or tubing 240c, 245c.

After adjusting the clamp or clip 708 to the second position to block flow through the lumens or tubing 240c, 245c, the force in the direction arrows A and/or B may be removed from the arms 716 and the arms 716 and the tubing 410 may return to the first position, as depicted in FIG. 9B. In some cases, the material of the tubing 410 may be sufficiently resilient to bias the tubing 410 and the arms 716 to the first position. Alternatively or additionally, the arms 716 may be biased by the material of and/or a biasing mechanism of the clamp or clip 708 to the first positions, such that when the force in the direction of arrows A and/or B is removed from the arms 716, the arms 716 return to the first position, but this is not required. When the arms 716 are biased to the first position, material of the tubing 410 may be configured to bias at least the tubing 410 to the first position depicted in FIG. 9B, but this is not required.

FIG. 10 depicts a schematic view of the flow control component 700 configured as a clamp or clip 708 that is a separate component from tubing or a connector of the endoscope system 200, 300, 400, where clamp or clip 708 is depicted in a side view and the tubing 410 is depicted in a cross-section view. The clamp or clip 708 in FIG. 10 is in the second position configured to block flow of fluid through the tubing 410 (e.g., through the gas supply tubing 240c and/or the lens wash tubing 245c) and is maintained in the second position with a coupler 718 maintaining the first arm 716a in a position relative to the second arm 716b. Although the clamp or clip 708 with the coupler 718 in FIG. 10 is depicted as a stand-alone clamp or clip 708, the clamps or clips 708 forming a part of tubing or a connector of the endoscope system 200 may include one or more couplers 718.

The coupler 718 may take on any suitable configuration. The coupler 718, may be or may include a ratchet component (e.g., a ratchet clip), a hook and loop connector, a belt connector, and/or other suitable connector configured to releasably maintain the first arm 716a in a position relative to the second arm 716b. In one example, as depicted in FIG. 10, the coupler 718 may be or include a ratchet clip having an extender 720 having a first end fixedly coupled to one of the first arm 716a and the second arm 716b (e.g., the second arm 716b in FIG. 10) and a second end releasably coupled to the other of the first arm 716a and the second arm 716b (e.g., the first arm 716a in FIG. 10). Although not required, the second end of the extender 720 may include a catch 722 configured to engage teeth 724 on one of the arms 716 (e.g., the first arm 716a), but this is not required, and the teeth 724 may be on the extender 720 with the catch 722 on the arm 716 and/or the coupler 718 may take on one or more other suitable configurations.

FIGS. 11A-C depict schematic views of a fluid control component 700 in the form of a clamp or clip 708 substantially similar to the configuration of the clamp or clip 708 depicted in FIGS. 9A-9C, but where the coaxial tubing 410 forms a bite valve 726. Further, although not depicted, the clamp or clip 708 may include a coupler, such as the coupler 718 depicted in and discussed with respect to FIG. 10 and/or other suitable coupler or engagement mechanisms.

The bite valve 726 may be a necked portion of the coaxial tubing 410 that is biased to a first position (e.g., closed configuration or relaxed position) that blocks flow through the gas supply tubing 240c and/or the lens wash tubing 245c (or other tubing disclosed herein). The second end 708b of the clamp or clip 708 may be configured to engage the coaxial tubing 410 at an exterior surface of the bite valve 726 and when force is applied by the second end 708b of the clamp or clip 708 to the bite valve 726, the bite valve 726 may adjust to a second position (e.g., an opened configuration or actuated position) and allow fluid to flow through the gas supply tubing 240c and/or the lens wash tubing 245c (e.g., through lumens of the tubing). Although the bite valve 726 and the clamp or clip 708 are depicted and discussed with respect to the coaxial tubing 410, it is contemplated that the bite valve 726 in only the gas supply tubing 240c, only the lens wash tubing 245c, the irrigation tubing 255c, the connector 290, and/or in one or more other suitable connectors and/or tubing of the endoscope system 200 and the clamp or clip 708 may be configured to engage the bite valve 726 at any suitable location within the endoscope system.

FIG. 11B depicts a schematic end view of the clamp or clip 708 on the connector 290 and a cross-section of the tubing 410 of FIG. 11A, taken along line 11B-11B. As depicted in FIG. 11B, the clip 708 and arms 716a and 716b are positioned in a first position such that the bite valve 726 is in a closed position preventing fluid flow through the tubing or the lumens thereof (e.g., the gas supply tubing 240c and the lens wash tubing 245c and/or other suitable tubing).

FIG. 11C depicts a cross-sectional view of the clamp or clip 708 on the connector 290 and the tubing 410, with the clamp or clip 708 in the second position acting on the bite valve 726 such that flow is allowed to flow through the lumens or tubing (e.g., the gas supply tubing 240c and the lens wash tubing 245c and/or other suitable tubing). In one example, a force in the direction of arrow A may be applied to the first arm 716a and/or in the direction of arrow B may be applied to the second 716b to adjust the clamp or clip 708 to the second position from the first position and allow a flow of fluid to pass through the lumens or tubing 240c, 245c.

After adjusting the clamp or clip 708 to the second position to allow the flow of fluid through the lumens or tubing 240c, 245c, the force in the direction arrows A and B may be removed from the arms 716 and the arms 716 and the tubing 410 may return to the first position, as depicted in FIG. 11B, to close the bite valve 726 and block a flow of fluid through the lumens or tubing 240c, 245c. In some cases, the material of the tubing 410 may be sufficiently resilient to bias the tubing 410 and the arms 716 to the first position. Alternatively or additionally, the arms 716 may be biased by the material of and/or a biasing mechanism of the clamp or clip 708 to the first position, such that when the force in the direction of arrows A and/or B is removed from the arms 716, the arms 716 return to the first position. When the arms 716 are biased to the first position, material of the tubing 410 may be configured to bias at least the tubing 410 to the first position depicted in FIG. 11B.

In some cases, the tubing and/or connectors of the endoscope system 200, 300, 400 may include a valve that is configured to block fluid flow through the connector and/or tubing when the connector and/or tubing is disconnected from a fluid port or other suitable connection point of the endoscope system 200, 300, 400, where the fluid port may be coupled to or is configured to be coupled to a fluid line of the endoscope 100. Then, when the connector and/or tubing including the valve connects to the fluid port or other suitable connection point of the endoscope system 200, 300, 400, the valve may be caused to open as a result of the connection to the fluid port and/or other suitable connection point and allow fluid to flow through the connector and/or tubing, across the valve, and into the fluid port or other suitable connection point.

The valve may be a passive flow prevention valve that defaults to a closed position and may be actuated to an opened position. The valve may be any suitable type of passive flow prevention valve configured to adjust from a first state or position (e.g., closed state or position) to a second state or position (e.g., an opened state or position) in response to engaging with a fluid port or connection point of the endoscope system 200. Example suitable valves include, but are not limited to, a check valve, a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check, a spring-loaded valve, flapper valve, a kinking system, and/or other suitable passive flow prevention valves.

In some cases, the valve may be an actively adjusted valve (e.g., a valve that may require active adjustment between an opened position and a closed position) incorporated into connectors and/or tubing of the endoscope system 200, 300, 400. In one example, the valve may include a threaded twist feature, which may be or may be similar to a touhy borst adapter or connector, incorporated into the tubing and/or connectors. The actively adjusted valve may be located proximate a connection portion of the connector and/or tubing and/or at one or more other suitable locations. The connectors and/or tubing of the endoscope system 200, 300, 400 may include only passive flow prevention valves, only actively adjusted valves, and/or a combination of passive flow prevention valves and actively adjusted valves.

In one example of an actively adjusted valve, a valve actuator may be rotated or otherwise adjusted in a first direction to close one or more lumens of the connector and/or the tubing. The valve actuator may then be rotated or otherwise adjusted in a second direction to open the one or more lumens of the connector and/or the tubing.

FIGS. 12A and 12B depict schematic view of an illustrative configuration of the fluid control component 700 that may be in the form of valves 728 incorporated into the connector 290, where the fluid control component 700 includes a valve 728 in each port 292 configured to couple with the fluid port 294 of and/or in fluid communication with an endoscope umbilical (e.g., the umbilical 260 via the connector 265). As depicted in FIGS. 12A and 12B, the valves 728 may be duck bill valves, but this is not required and the valves 728 may be other suitable types of valves configured to block flow in at least one direction when in a first position and adjust to a second position at which fluid may flow across the valve in two directions when connected with a fluid port 294. Although FIGS. 12A and 12B depict a valve 728 in each of the ports 292, the valve 728 may be located in less than all of the ports 292 (e.g., located in only the first port 292a or only the second port 292b).

In some cases, the connector 290 may be formed from a first component 296 and a second component 298. In one example, the first component 296 may be a core or a support structure and the second component 298 may be a device coupling member that is resilient and configured for coupling with the fluid ports 294.

The portion of the ports 292 formed from the second component 298 may include one or more features configured to facilitate engaging the fluid ports 294. For example, the ports 292 may include features such as coupling portions 299 (e.g., ribs, ridges, etc.) configured to engage a male feature of the fluid ports 294 of the endoscope system 200, 300, 400. The coupling portions 299 may be incorporated into or may be the valves 728 and/or may be separate from the valves 728.

The valves 728 incorporated into one or more ports 292 of the connector 290 may be formed from one or more materials that are configured to resiliently adjust from a first position (e.g., a closed and/or resting position) to a second position (e.g., an opened or actuated position) in response to engagement with a fluid port 294 and then, back to the first position in response to disengagement from the fluid port 294. Example suitable material for forming the valves 728 include, but are not limited to, thermoplastic elastomer (TPE), styrene-ethylene-butylene (SEB), rubber, silicone, and/or other suitable elastomeric and/or resilient materials.

FIG. 12A depicts the first port 292a and the second port 292b disconnected from a first fluid port 294a and a second fluid port 294b of an endoscope umbilical, where the valves 728 in the first port 292a and the second port 292b are in a first position (e.g., a closed or relaxed position and the valves 728 may be biased to the first position) that results in blocking a flow of fluid through the lens wash tubing 245c and through the gas supply tubing 240c. Fluid forces acting on the valves 728 (e.g., water moving from the water reservoir 270, 305, 405 to the first port 292a) may not be sufficient to open the valves and thus, the valves 728 block fluid flow therethrough.

To open the valves 728, the connector 290 may be coupled with the fluid ports 294, such that the first fluid port 294a is received within the first port 292a and/or the second fluid port 294b is received within the second port 292b. The received fluid ports 294a, 294b may engage the valves 728 in the first port 292a and the second port 292b, which may result in adjusting the valves 728 from the first position to the second position at which fluid may flow across the valves 728 and between the lens wash tubing 245c and the first fluid port 294a and between the gas supply tubing 240c and the second fluid port 294b, represented by arrows F1 and F2, respectively, depicted in FIG. 12B. The fluid ports 294 may be formed from any suitable types of materials of sufficient rigidity to adjust the valves 728 from the first position to the second position in response to engaging the valves 728.

Further, after connecting the connector 290 with the fluid ports 294, the connectors 290 may be separated from the fluid ports 294. Once the fluid ports 294 separate from or otherwise disengage the valves 728, the valves 728 may automatically return to the first position due to a bias force of the configuration of the valves 728 (e.g., due to a resilient nature of the material forming the valve 728, a shape of the valves 728, a biasing component acting on the valves 728, and/or due to one or more other suitable configurations or features of the valve 728).

FIGS. 13A and 13B depict a configuration the fluid control component 700 that may be in the form of a valve 728 incorporated into the connector 1390 coupled to the irrigation supply tubing 255c, where the connector 1390 includes a port 1392 configured to couple with the fluid port 1394 of and/or in fluid communication with an endoscope umbilical (e.g., the umbilical 260 via the connector 265). As depicted in FIGS. 13A and 13B, the valve 728 may be a spring-loaded duck bill valve, but this is not required and the valve 728 may be other suitable type of valve configured to block flow in at least one direction when in a first position and adjust to a second position at which fluid may flow across the valve in desired directions when connected with the fluid port 1394.

In some cases, the valve 728 may include a closure component 730 (e.g., a flapper, lid, cap, and/or other suitable closure mechanism) coupled to a biasing mechanism 732 (e.g., a spring and/or other suitable biasing mechanism). The biasing mechanism 732 may have a spring constant sufficient to cause the closure component 730 to remain closed over a port 1392 when irrigation fluid (e.g., represented by arrows F3) is acting against the closure component 730, as depicted in FIG. 13A.

To open the valve 728 or adjust the valve 728 from the first position (e.g., a closed or resting position) to the second position (e.g., an opened and/or actuated position), the connector 1390 may be coupled with the fluid port 1394, such that the fluid port 1394 may receive the port 1392 of the connector 1390, as depicted in FIG. 13B. In some cases, the connector 1390 may include threads 1391 configured to engage threads 1395 of the fluid port 1394, which may facilitate coupling the connector 1390 to the fluid port 1394 and overcoming a biasing force of the biasing mechanism 732. Other suitable configurations, however, are contemplated for coupling the connector 1390 to the fluid port 1394 including, but not limited to, a friction fit configuration, a latch configuration, etc. When the connector 1390 is coupled with the fluid port 1394, the force of the coupling may act on the biasing mechanism 732 and against the bias force thereof to compress the biasing mechanism 732 and cause the closure component 730 to lift off of the port 1392 to allow fluid to flow across the valve 728 from the irrigation supply tubing 255c into the fluid port 1394.

Further, after connecting the connector 1390 with the fluid port 1394, the connector 1390 may be separated from the fluid port 1394. In response to separating the fluid port 1394 from the connector 1390, the valve 728 may automatically return to the first position due to a bias force of the biasing mechanism 732 acting on the closure component 730 and causing the closure component 730 to close off the port 1392 and block the flow of irrigation fluid from a water reservoir.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A tubing assembly for connecting to a fluid line of an endoscope and a fluid source, the tubing assembly comprising:
an elongate tube, the elongate tube having a lumen;
a connector coupled to an end of the elongate tube, the connector having a port in fluid communication with the lumen; and
a fluid control component configured to selectively adjust fluid flow through the lumen and the port.

2. The tubing assembly of claim 1, wherein the fluid control component is configured to selectively depressurize the lumen.

3. The tubing assembly of claim 1, wherein the fluid control component is a valve configured to selectively adjust fluid flow through the lumen.

4. The tubing assembly of claim 3, further comprising:
a cover configured to couple to the fluid source; and
wherein the valve is in fluid communication with the fluid source through the cover and the valve is configured to be selectively actuated from a location exterior of the fluid source.

5. The tubing assembly of claim 3, wherein the valve is in fluid communication with the lumen through the connector and the valve is configured to be selectively actuated from a location exterior of the connector.

6. The tubing assembly of claim 3, wherein the valve is in the port and is configured to be actuated in response to the port coupling with an umbilicus of an endoscope.

7. The tubing assembly of claim 3, wherein the valve is biased to a closed position and configured to adjust from the closed position to an opened position in response to coupling the connector with fitting of the endoscope.

8. The tubing assembly of claim 1, wherein the fluid control component includes a clamp configured to selectively engage the elongate tube.

9. The tubing assembly of claim 8, wherein:
the elongate tube includes a bite valve in communication with the lumen and the bite valve blocks fluid flow through the lumen when in a resting configuration; and
the clamp is configured to engage the bite valve through the elongate tube to adjust the bite valve to an open configuration that allows fluid to flow through the lumen.

10. The tubing assembly of claim 8, wherein the clamp is configured to block fluid flow through the lumen when engaging the elongate tube.

11. The tubing assembly of claim 1, wherein:
the lumen is a first lumen and the elongate tube comprises a second lumen;
the first lumen and the second lumen are coaxial; and
the fluid control component is configured to selectively adjust fluid flow through the first lumen, the second lumen, and the port.

12. A tubing assembly for connecting to a fluid line of an endoscope and a fluid source, the tubing assembly comprising:
an elongate tube, the elongate tube having a lumen;
a connector coupled to an end of the elongate tube, the connector in fluid communication with the lumen;
a port in fluid communication with the lumen, the port is configured to couple to the fluid line of the endoscope; and
a valve at the connector and in fluid communication with the lumen, the valve is configured to adjust between a closed position at which fluid through the valve is blocked and an opened position at which fluid flows through the valve.

13. The tubing assembly of claim 12, wherein the valve is configured to vent fluid to atmosphere when in the opened position and the connector is coupled to the endoscope or the fluid source.

14. The tubing assembly of claim 12, wherein the valve is configured to fluidly connect the fluid line of the endoscope to the lumen when the valve is adjusted from the closed position to the opened position.

15. The tubing assembly of claim 12, wherein the valve is biased to the closed position.

16. A tubing assembly for connecting to a fluid line of an endoscope and a fluid source, the tubing assembly comprising:
an elongate tube, the elongate tube having a lumen;
a connector coupled to an end of the elongate tube, the connector in fluid communication with the lumen;
a port in fluid communication with the lumen, the port is configured to couple to the fluid line of the endoscope; and
a clamp configured to engage the elongate tube.

17. The tubing assembly of claim 16, wherein:
the elongate tube includes a bite valve in communication with the lumen and the bite valve blocks fluid through the lumen when in a resting configuration; and
the clamp is configured to engage the bite valve through the elongate tube to adjust the bite valve to an opened configuration that allows fluid to flow through the lumen.

18. The tubing assembly of claim 16, wherein the clamp is configured to block fluid flow through the lumen when engaging the elongate tube.

19. The tubing assembly of claim 16, wherein the clamp includes a ratchet component configured to releasably lock the clamp in a position relative to the elongate tube.

20. The tubing assembly of claim 16 wherein the clamp extends from the connector and is configured to engage the elongate tube at a location proximal of the connector.
